(19) United States
(12) Patent Application Publication
(10) Pub. No.: US 2025/0188527 A1
(43) Pub. Date: Jun. 12, 2025

(54) METHOD AND DEVICE FOR EXTRACTING AND/OR AMPLIFYING A TARGET NUCLEIC ACID

(71) Applicant: HP Health Solutions Germany GmbH, Grafelfing (DE)

(72) Inventors: Joachim Stehr, Martinsried (DE); Federico Buersgens, Martinsried (DE); Cecilia Rebuffo-Scheer, Martinsried (DE); Bettina Grotz, Martinsried (DE); Juliane Duczek, Martinsried (DE); Simon Schmidbauer, Martinsried (DE)

(73) Assignee: HP Health Solutions Germany GmbH, Grafelfing (DE)

(21) Appl. No.: 18/012,615

(22) PCT Filed: Jun. 22, 2021
(86) PCT No.: PCT/EP2021/067049

§ 371 Date: Jun. 18, 2024

(30) Foreign Application Priority Data
Jun. 26, 2020 (DE) 10 2020 116 930.2

(51) Int. Cl. C12Q 1/6844; B01L 3/00; B01L 7/00

(52) U.S. Cl. CPC C12Q 1/6844; B01L 3/502761; B01L 7/52; B01L 2200/0668; B01L 2300/0858; B01L 2300/1827; B01L 2400/043

(57) ABSTRACT

Provided is a method for extracting a target nucleic acid (12) from a sample fluid (30). The method comprises providing a sample fluid (30) comprising the target nucleic acid (12) in a reaction container (22) and providing magnetic microparticles (10) in the sample fluid (30), each functionalized with at least one extraction nucleic acid (16), wherein the extraction nucleic acids (16) are at least partially complementary to the target nucleic acid (12). Further, the method comprises hybridizing at least a part of the target nucleic acid (12) with one of the extraction nucleic acids (16) and binding the target nucleic acid (12) via the extraction nucleic acid (16) to one of the magnetic microparticles (10). Furthermore, the method comprises providing a magnetic field in the reaction container (22) in such a manner that at least a part of the magnetic microparticles (10) associated with the target nucleic acid (12) sediments on a container wall (22a, 22b, 22c) of the reaction container (22). Further provided are devices and methods for amplifying a target nucleic acid (12), using magnetic microparticles (10) for extraction of a target nucleic acid (12), and a magnetic microparticle.

Methods for extracting and/or amplifying a target nucleic acid, a device for amplifying a target nucleic acid and a use of magnetic microparticles for extracting a target nucleic acid are provided. Thus, the embodiments are particularly in the field of molecular diagnostics.

In molecular diagnostics, methods are known by means of which the presence or absence of target nucleic acids in a sample to be examined can be determined. However, these methods have limited sensitivity and therefore often require a minimum concentration of target nucleic acid present to reliably determine its presence. It is therefore often necessary to first extract the target nucleic acid, if present, from a sample fluid prior to an amplification reaction to be performed for detection, for example by means of a PCR.

Alternatively or additionally, an attempt can be made to concentrate the target nucleic acid in that area of the reaction solution in which the amplification reaction primarily occurs. For example, DE 44 09 436 A1 discloses a method in which the target nucleic acids are chemically and/or physically bound to a heating element by functionalizing the heating element or moving the target nucleic acids to the heating element by means of magnetic particles.

It is an object to provide a method and a device suitable for efficient extraction and/or amplification of a target nucleic acid, thereby improving the detection limit for the detection of nucleic acids.

This object is solved by methods, devices and uses having the features of the respective independent claims. Advantageous embodiments are given in the subclaims and in the description.

An embodiment relates to a method for extracting a target nucleic acid from a sample fluid. The method comprises providing a sample liquid having the target nucleic acid in a reaction container, and providing magnetic microparticles in the sample liquid each functionalized with at least one extraction nucleic acid, wherein the extraction nucleic acids are at least partially complementary to the target nucleic acid. In addition, the method comprises hybridizing at least part of the target nucleic acid with one of the extraction nucleic acids and binding the target nucleic acid to one of the magnetic microparticles via the extraction nucleic acid. Optionally, hybridizing may comprise annealing at least a part of the sample liquid in such a manner that the target nucleic acid hybridizes with one of the extraction nucleic acids and binds to one of the magnetic microparticles via the extraction nucleic acid. Further, the method comprises providing a magnetic field in the reaction container in such a manner that at least a part of the magnetic microparticles associated with the target nucleic acid attaches to an extraction element disposed in and/or on the reaction container.

A further embodiment relates to a method for amplifying a target nucleic acid. The method comprises providing magnetic microparticles in a reaction solution, each of which is functionalized with at least one primer and is connectable or linked to at least one target nucleic acid via the at least one primer, and providing a local heating element in direct contact with the reaction solution. In addition, the method comprises exposing at least a part of the magnetic microparticles associated with the target nucleic acid in the reaction solution to a magnetic field in such a manner that at least a part of the magnetic microparticles attach to the local heating element, and locally heating the reaction solution at a denaturation temperature by means of the local heating element in the area where the magnetic microparticles attach to the local heating element.

A further embodiment relates to a method for amplifying a target nucleic acid. In this regard, the method comprises the following steps:
a) providing a sample fluid containing the target nucleic acid (12) in a reaction container and at least one local heating element in direct contact with the sample fluid;
b) providing magnetic microparticles in the sample fluid, wherein the magnetic microparticles are each functionalized with at least one primer for amplifying the target nucleic acid;
c) hybridizing the target nucleic acid with at least one of the primers functionalized on the magnetic microparticles;
d) providing a magnetic field in the reaction container in such a manner that at least a part of the magnetic microparticles with the target nucleic acid hybridized thereto attaches to the local heating element;
e) removing the sample fluid from the reaction container;
f) providing a reaction solution for performing an amplification reaction of the target nucleic acid in the reaction container;
g) locally heating the reaction solution to a temperature in a denaturation temperature by means of the at least one local heating element in the area where the magnetic microparticles are attached to the local heating element.

A further embodiment relates to a device for amplifying a target nucleic acid. The device comprises a reaction container designed to receive a reaction solution containing the target nucleic acid, and at least one local heating element arranged in and/or on the reaction container in such a manner that the local heating element is at least partially in direct contact with the reaction solution when the reaction container is filled with reaction solution. Further, the device comprises a magnet for generating a magnetic field, wherein the magnetic field acts on at least a part of the magnetic microparticles present in the reaction solution in such a manner that they attach to the local heating element.

A further embodiment relates to a use of magnetic microparticles for extracting a target nucleic acid from a sample fluid.

A further embodiment relates to a magnetic microparticle functionalized with at least one primer for an amplification reaction of a target nucleic acid.

Certain embodiments provide the advantage that reliable hybridization of the target nucleic acid present in the sample fluid with the extraction nucleic acids functionalized to the microparticles can be achieved during extraction of the target nucleic acid from the sample fluid, since a low average distance of the microparticles with the target nucleic acids present in the sample fluid can be achieved due to the microparticles being in suspension in the sample fluid. In particular, some embodiments thereby offer the advantage that the central spacing of the magnetic microparticles in the sample fluid and, concomitantly, the central spacing of a magnetic microparticle to a target nucleic acid can be determined by a suitable choice of the concentration of the microparticles in the sample fluid.

In addition, certain embodiments offer the advantage that this efficient hybridization can be combined with an efficient means of separating the target nucleic acid from the rest of the sample fluid, since by providing a magnetic field and corresponding magnetic forces, the magnetic microparticles can be targeted and moved in a desired direction in the reaction container, while the rest of the sample fluid or reaction solution is unaffected or virtually unaffected by this. In this manner, the magnetic microparticles and, correspondingly, the target nucleic acids functionalized and hybridized to them can be attached to a container wall, while the remaining sample fluid remains unaffected in the reaction container and can be removed therefrom.

Further, some embodiments offer the advantage that even during amplification, the target nucleic acids hybridized to the functionalized magnetic microparticles can be easily moved into the area of local heating. Here, too, this can be achieved by means of a magnetic field and the magnetic forces thereby acting on the magnetic microparticles, while the reaction solution remains unaffected. Thus, the magnetic microparticles can optionally be used in multiple beneficial ways for detecting a target nucleic acid, namely, on the one hand, for efficiently extracting the target nucleic acids from a sample fluid and, on the other hand, for efficiently concentrating the target nucleic acids in the area that is locally heated in the course of an amplification reaction.

Magnetic microparticles are microparticles which have ferromagnetic or paramagnetic properties. The size of the microparticles is optionally in a range from about 10 nm up to about 2 mm, optionally in a range from 100 nm to 1 mm, optionally in a range from 500 nm to 50 μm. The shape of the microparticles is freely selectable and can, for example, be spherical, cubic, cuboidal or ellipsoidal. Optionally, the magnetic microparticles having ferromagnetic properties are formed of or include at least one of the following materials: iron, nickel, cobalt, AlNiCo, SmCo, Nd₂Fe₁₄B, Ni₈₀Fe₂₀ (“permalloy”), and/or NiFeCo alloys. Optionally, the magnetic microparticles with paramagnetic properties are formed of or include at least one of the following materials: alkaline earth metals, alkali metals, and/or rare earths. Alternatively, a magnetic microparticle may be formed of a non-magnetic material, such as glass and/or silicate, with magnetic materials embedded therein. For example, such a microparticle may have a core of magnetic materials. In this regard, the magnetic microparticles are optionally provided with one or a plurality of coatings to enable or promote functionalization with nucleic acids, in particular with extraction nucleic acids and/or primers. Optionally, at least one extraction nucleic acid and/or primer and a maximum of 1012 extraction nucleic acids and/or primers are functionalized on a microparticle. Optionally, a surface density of extraction nucleic acids and/or primers functionalized to the surface of a magnetic microparticle is in an area of 0.0001 to 1 per square nanometer. Optionally, the microparticles may have a coating that enables and/or facilitates functionalization with oligonucleotides. For example, the magnetic microparticles may be at least partially functionalized with streptavidin at their surface.

The sample fluid is optionally a starting fluid containing the target nucleic acid (e.g., sample material containing the target nucleic acid, or sample material in which the target nucleic acid has been (previously) released (e.g., from pathogens and/or cells), or a fluid in which the nucleic acid is already present in the purified state). The sample fluid may optionally also have or be provided with reagents that promote hybridization of the target nucleic acid to the functional nucleic acid or extraction nucleic acid or primer oligonucleotide on the local heating element, such as appropriate salts.

The extraction element can be a part of the reaction container and/or be firmly connected to the reaction container, or be independent and separate from the reaction container. Optionally, for example, a container wall and/or the bottom and/or the lid of the reaction container may constitute an extraction element. Alternatively, the extraction element may be formed by a separate device, such as one or more wires and/or a foil, which may be disposed in the reaction solution. For example, the extraction element can be attached in and/or to the reaction container. Optionally, the extraction element may be at least partially ferromagnetic to attract the magnetic microparticles.

The fact that the magnetic microparticles are attached to the extraction element means that they are arranged on a surface of the extraction element and are no longer freely suspended in the reaction solution or sample fluid. For example, the microparticles attaching to the extraction element or to the local heating element could sediment on the extraction element or on the local heating element, respectively, wherein the sedimentation is not necessarily caused exclusively or primarily by the effect of gravity, but is primarily caused by the magnetic field provided. The attached microparticles are optionally firmly connected to the extraction element or the local heating element in such a manner that they remain at least partially attached to the extraction element or local heating element even when the reaction solution and/or the sample fluid are removed.

Optionally, a container wall may be formed as an extraction element. The container wall of the reaction container to which the microparticles attach during extraction of the target nucleic acid may differ from the container wall of the reaction container to which the microparticles attach during amplification of the target nucleic acid. Optionally, during extraction of the target nucleic acid, the microparticles do not attach to that container wall that has the local heating element or is formed as the local heating element. However, during amplification of the target nucleic acid, the magnetic microparticles are to be attached to the container wall that has the local heating element or is designed as such. The optional attachment to different container walls during extraction on the one hand and amplification on the other hand offers the advantage of avoiding the formation of unwanted deposits on the local heating element during extraction of the target nucleic acid from the sample fluid, which could otherwise lead to impairment of local heating and/or performance of the PCR. However, according to an optional embodiment, attachment of the extracted microparticles directly to the local heating element is advantageous for the amplification reaction to be performed.

The sample fluid is optionally a fluid optionally having the target nucleic acid and also comprising other components. In particular, the sample fluid may have other components that should not be extracted as well. These other components may comprise other nucleic acids, i.e., nucleic acids that have a different nucleotide sequence. The other components may also have impurities that are of a non-nucleotide nature. In particular, the sample fluid may be a fluid that is, for example, of human and/or animal and/or plant and/or other organic origin. For example, the sample fluid may contain or consist of blood and/or secretions and/or body excretions and/or secretions from mucous membranes and/or saliva and/or cellular fluid. The sample fluid may optionally have been subjected to one or more treatments prior to extraction, for example to at least partially release nucleic acids contained in the sample fluid. For example, the sample fluid may have been subjected to a treatment to lyse or disrupt cells therein, for example, in order to at least partially release the nucleic acids that may be present therein from the cells, in such a manner that the nucleic acids are optionally present freely in the sample fluid and are at least partially not or no longer enclosed in cell nuclei and/or cells. Optionally, lysing is performed in such a manner that the released nucleic acids are at least not completely destroyed and, particularly optionally, are completely preserved or remain intact.

In particular, the sample fluid may optionally be present in such a manner that performing an amplification reaction to amplify the extracted nucleic acid in the sample fluid is not possible. For example, the sample fluid may have physical and/or chemical and/or biological properties that prevent an amplification reaction, such as PCR, from being performed in the sample fluid. For example, the sample fluid may have a viscosity and/or pH and/or salt concentration and/or polarity and/or enzymes and/or proteases that do not allow an amplification reaction to be performed, for example because the activity of the polymerase enzymes required for this is inhibited or enzymes such as proteases are present that can degrade the polymerase enzymes.

The sample fluid is of such a nature that the magnetic microparticles can be suspended in the sample fluid and hybridization of single-stranded target nucleic acids to the primers or extraction nucleic acids functionalized on the magnetic microparticles is made possible.

Optionally, at least one parameter of the sample fluid may be adjusted to allow hybridization of the target nucleic acid to the functional nucleic acid given the level of complementarity present. For example, a concentration of MgCl₂ in the sample fluid can be increased to allow hybridization even at low levels of complementarity, whereas optionally the concentration of MgCl₂ in the sample fluid can be decreased to allow hybridization only at a certain higher level of complementarity.

The reaction solution optionally has reagents necessary for polymerase chain reaction amplification and can be brought into contact with the local heating element and the magnetic microparticles in such a manner that the magnetic microparticles are suspended in the reaction solution.

The reaction solution is optionally a fluid in which the target nucleic acid can survive as such in single-stranded and/or double-stranded form and/or is stabilized. However, the reaction solution is different from the sample fluid. In other words, the reaction solution is optionally designed in such a manner that the target nucleic acid is not damaged by the interaction with the reaction solution. For example, the reaction solution may be present as an aqueous solution and/or as a buffer solution. If amplification of the target nucleic acid is intended after extraction of the target nucleic acid, the reaction solution may optionally be designed in such a manner that the reaction solution enables such amplification solution to be performed. For example, the reaction solution may be formed as a buffer solution in which it is possible to perform PCR to at least partially amplify the target nucleic acid. The reaction solution is optionally provided in a reaction container. The provided reaction solution optionally has a volume of at least 1 μl and not more than 10 ml, more optionally of at least 5 μl and not more than 1 ml, most optionally of at least 10 μl and not more than 100 μl.

In the context of the present disclosure, the terms “nucleic acid” and “oligonucleotide” comprise not only (deoxy)-ribonucleic acids or (deoxy)-oligo ribonucleotides, even if the aforementioned are optional, but also nucleic acids and oligonucleotides that contain one or more nucleotide analogs with modifications at their backbone (for example, methyl phosphonates, phosphothioates or peptide nucleic acids (PNA), in particular at a sugar of the backbone (for example, 2′-alkyl derivatives, 3′- and/or 5′-aminoriboses, locked nucleic acids [LNA], hexitol nucleic acids, morpholinos, glycol nucleic acid (GNA), threose nucleic acid (TNA), or tricyclo-DNA, compare the paper by D. Renneberg and C. J. Leumann, “Watson-Crick base-pairing properties of Tricyclo-DNA,” J. Am. Chem. Soc., 2002, vol. 124, pages 5993-6002, the contents of which are incorporated herein by reference) or which contain base analogs, for example, 7-deazapurines or universal bases such as nitroindole or modified natural bases such as N4-ethyl cytosine. In an embodiment, the nucleic acids or oligonucleotides are conjugates or chimeras with non-nucleoside analogs, for example, PNA. In an embodiment, the nucleic acids or oligonucleotides contain non-nucleoside and/or non-nucleotide moieties such as spacers, for example hexaethylene glycol or Cn spacers with n between 3 and 6, at one or more positions. If the nucleic acids or oligonucleotides contain modifications, these are selected in such a manner that hybridization with natural DNA/RNA analytes is also possible with the modification. Optional modifications influence the melting behavior, optionally the melting temperature, in particular to be able to distinguish hybrids with different degrees of complementarity of their bases (mismatch discrimination). Optional modifications comprise LNA, 8-aza-7-deaza-purines, 5-propynyl uracil and cytosine, and/or abasic breaks or modifications in the nucleic acid or oligonucleotide. Further modifications according to the disclosure include, for example, modifications with biotin and/or thiol and/or sulfur and/or fluorescent donor and fluorescent acceptor molecules and/or quenchers.

The terms target nucleic acid and nucleic acid are used as equivalents throughout this document unless otherwise noted. In this context, the nucleic acid being extracted from the sample fluid means that the nucleic acid is at least partially isolated in and/or from the sample fluid and can optionally be separated from the sample fluid. The nucleic acid to be extracted from the sample fluid may also be referred to as target nucleic acid or target nucleic acid. In particular, extracting a nucleic acid may thus comprise or serve to separate the nucleic acid from the sample fluid. The extraction of the nucleic acid can be designed in such a manner that only the nucleic acid to be extracted is extracted from the sample fluid or that other components of the sample fluid are also extracted, such as other nucleic acids. Particularly optionally, however, only the nucleic acid to be extracted is extracted from the sample fluid, in such a manner that, in particular, other nucleic acids and other components of the sample fluid are not extracted as well, but remain in the sample fluid. Optionally, after extraction of the nucleic acid, the concentration and/or number of copies of the extracted nucleic acid in the sample fluid is lower than before extraction, if at all the nucleic acid to be extracted was present in the sample fluid before extraction.

A higher degree of complementarity between the extraction nucleic acid and the target nucleic acid may offer advantages in terms of selectivity or specificity during extraction. Thus, a high degree of complementarity between the extraction nucleic acid and the target nucleic acid may allow substantially only the target nucleic acid to bind to the extraction nucleic acid, while binding of other nucleic acids from the sample fluid to the functional nucleic acid is an exception. On the other hand, a low degree of complementarity between the extraction nucleic acid and the target nucleic acid may optionally allow binding of different nucleic acids from the sample fluid to the extraction nucleic acid, in such a manner that other nucleic acids can also be extracted from the sample fluid and/or the target nucleic acid can also be extracted, if the extraction nucleic acid has only a low degree of complementarity to it, for example because the base sequence of the target nucleic acid is not known to a sufficient degree and accordingly the extraction nucleic acid cannot be matched exactly to the target nucleic acid.

The fact that a magnetic microparticle is functionalized with an extraction nucleic acid and/or a primer means that the extraction nucleic acid or the primer is bound to the microparticle. This in turn means that the extraction nucleic acid or primer is mechanically firmly attached to the microparticle, in particular by a chemical and/or electrostatic bond. For example, the extraction nucleic acid or primer may be attached to a surface of the microparticle by means of one or more thiol bonds and/or sulfur bonds. Optionally, for this purpose, the microparticle is at least partially provided on its surface with a material that allows the binding of nucleic acids. For example, a gold-plated surface can be used to bind the extraction nucleic acid and optionally other nucleic acids to the local heating element via one or more thiol and/or sulfur bonds. For example, streptavidin-biotin binding can also be used to bind the extraction nucleic acid or primer and/or other nucleic acids to the microparticle if, for example, optionally one of the two partners (streptavidin or biotin) has been previously bound to the microparticle and the functional nucleic acid (optionally at the 5′ end) is modified with the other of the two partners and subsequently bound to the microparticle thereover. Other modifications, such as amino or carboxy groups, can also be used to bind the extraction nucleic acid or primer to the microparticle, and the surface of the local heating element can be modified for this purpose, for example, optionally beforehand, with epoxy and/or a metal. Optionally, binding occurs such that the 5′ end of the functional nucleic acid is bound to the local heating element, leaving 3′ end free. This can be particularly advantageous if the extraction nucleic acid itself is designed to serve as a primer in an amplification reaction to be performed after extraction.

Having the local heating element in contact with the sample fluid or reaction solution may comprise having a heating surface of the local heating element, such as a metal foil, in direct contact with the reaction solution or sample fluid. Alternatively, one or more protective layers may be arranged between the heating surface of the local heating element and the reaction solution or sample fluid, wherein the one or more protective layers have a very high themal conductivity and are optionally designed to be as thin as possible.

A local heating element is a heating element that is suitable for local heating of an area in the immediate vicinity of the local heating element. Local heating as defined in the disclosure is explained in detail below.

A polymerase chain reaction or PCR as defined in the present disclosure is a method for amplifying target nucleic acids in which a amplification cycle consisting of the steps of denaturation, hybridization and elongation is repeatedly run, optionally in that order. In each run, the number of nucleic acid molecules and, in particular, target nucleic acids can be increased (typically doubled in the best case), resulting in an exponential increase in the number of nucleic acid molecules. In the following, a target nucleic acid to be amplified is referred to as an “original”. The original is a single strand and can form a double strand together with its complementary strand, which is called a “complement”. The original and also the complement can be part of a larger nucleic acid. In particular, in a PCR, a copy of the original formed in one run of the amplification cycle may be a template for forming a complement in a subsequent run, and a copy of the complement formed may be a template for forming an original in a subsequent run. A common designation for the amplification product is “amplicon”.

The denaturation step is used to denature a nucleic acid double strand, that is, to separate it into its two single strands. For example, the original can be separated from the complement in the denaturation step. An optional type of denaturation is thermal denaturation (also referred to as “melting”). For this purpose, at least a part of the nucleic acid double strand or the whole double strand is exposed to a temperature, referred to as “denaturation temperature”, which causes or at least promotes a separation of the nucleic acid double strands. On the one hand, the optional denaturation temperature is selected in such a manner that nucleic acid double strands can be separated. On the other hand, the optional denaturation temperature is chosen low enough to avoid significant damage to a DNA polymerase that may also be present in the sample. A typical value for the denaturation temperature is 95° C.

To facilitate the following explanation of the disclosure, “denaturation step” in the nomenclature of the present disclosure means the step of the method in which the local heating element generates heat to heat the reaction volume in the immediate vicinity of the local heating element, thereby causing denaturation of double-stranded nucleic acid molecules. Accordingly, the duration of the denaturation step is the sum of the time during which the local heating element generates heat in the run of the cycle of the PCR relating to the denaturation step. Thus, in the case of a heating resistor and/or an inductive heating element as a heating device and/or as a local heating element, the duration of the denaturation step is the duration of a current passage through the heating device and/or through the local heating element to heat the reaction volume and thereby cause denaturation of double-stranded nucleic acid molecules. If the heating device or local heating element generates the heat in one run of the duplication cycle instead of in a plurality of separate time intervals, the duration of the denaturation step is the sum of the durations of these intervals. In particular, the denaturation step thus defined does not include a release of heat due to the inherent heat capacity of the local heating element, nor does it include the decay of the temperature in the part of the reaction volume adjacent to the local heating element, even if the temperatures present there are still within the area required for denaturation. In particular, this means that in the method according to an embodiment, denaturation can still take place after the denaturation step defined in this manner. It also means that the heat released to the reaction volume in the denaturation step is usually less than the heat generated in the denaturation step. However, since the heat capacity of the local heating device is negligible in a particularly optional embodiment, the heating duration optionally corresponds to the duration of the denaturation step.

The heating of the local heating element(s), which are optionally designed as resistive local heating elements and particularly optionally as metal foils, can optionally be achieved by means of short electrical pulses with which the local heating element(s) are energized. Particularly optionally, this is done in such a manner that only the immediate vicinity of the local heating element(s) is heated locally for a short time, optionally to perform denaturation of the nucleic acid molecules in the reaction volume, while the bulk of the reaction volume, i.e., the reaction solution, remains at a (in this sense “global”) base temperature at which, in particular, elongation, optionally also hybridization, can take place. This is optionally achieved by ensuring that the duration of heating by the heating device is so short that the heat field generated in the surrounding reaction volume can propagate only a few micrometers, thus creating a heating zone that optionally comprises only a tiny fraction of the reaction volume. In particular, this allows the amount of heat introduced to be so small that no substantial global heating of the reaction volume occurs.

The “global temperature” in the sense of the present disclosure is the average temperature of the reaction volume or the reaction solution in which the PCR occurs, based on the volume of the room, i.e., the temperature that occurs or would occur after thermalization of the reaction volume. “Global heating” is the increase in global temperature so defined.

Furthermore, by means of local heating it is achievable that after heating, particularly in the denaturation step, the introduced heat, which spreads from the heating zone into the rest of the reaction volume, causes only a negligible global temperature increase there. “Negligible” as used herein means, in particular, that the temperature increase is optionally too small to cause denaturation of the nucleic acid molecules and, particularly optionally, that the temperature increase is too small to interfere with hybridization and elongation.

In the following, local heating in the sense of the disclosure will be explained in more detail. When a current flows through a local heating element, which may be a metal foil with a thickness of about 20 μm or less, the local heating element starts to heat up at the beginning of the heating pulse. The local heating element is optionally designed to have the largest possible surface in contact with the reaction solution and, at the same time, the smallest possible heat capacity.

To realize the lowest possible heat capacity, the local heating element is optionally designed to have a thickness of less than 100 μm in at least one dimension, optionally less than 50 μm, and particularly optionally less than 30 μm. Such a limitation in one dimension could be, for example, a foil; if the limitation is in two dimensions, the local heating element could be, for example, a wire, and if the limitation is in three dimensions, for example, a sphere.

In order to make the local heating element not too fragile, it is useful that the material thickness in each dimension is at least 100 nm, optionally 1 μm and optionally 5 μm or 10 μm.

Particularly optionally, the heating device is designed in such a manner that the material has a magnetic permeability of optionally greater than 1, optionally greater than 1.1, optionally greater than 2, optionally greater than 5, optionally greater than 10, optionally greater than 20, particularly optionally greater than 50, optionally greater than 100, in such a manner that at a given magnetic field strength (e.g. by an external magnet) a high magnetic flux density results at the surface of the local heating element, which can be used to attract the magnetic particles. On the one hand, this allows the use of a relatively weak magnetic field to achieve a high magnetic flux density and, on the other hand, a targeted force effect on the magnetic microparticles towards the local heating element.

Optionally, the local heating element is formed of a metallic material. Optionally, the local heating element is formed of ferromagnetic materials such as steel and/or stainless steels and/or nickel and/or highly conductive nonferrous metals, such as brass and/or copper. Alternatively or additionally, the local heating element is at least partially formed of very hard materials, such as tungsten, which allow very thin designs of the local heating element. In addition, the local heating element optionally has a very high thermal conductivity. Optionally, the local heating element is designed in such a manner that it heats approximately homogeneously over the duration of the heating pulse. At the surface of the local heating element, which is in contact with the reaction solution during PCR, heat is transferred from the local heating element to the reaction solution, where it spreads to an increasingly larger volume. The propagation of a heat field occurs in the reaction solution by heat diffusion, for which the following rooted path-time law applies:

$$d \approx \sqrt{D \cdot t}$$

Where d represents the distance a heat front travels after a time t along a spatial direction in a reaction solution with thermal diffusivity D. This distance d is referred to in the following as the heat diffusion distance. That is, the heat generated in the local heating element can diffuse widely in the reaction solution with a typical temperature diffusivity (also known as thermal diffusivity) of D≈1.6·10⁻⁷ m²/s in the order of d≈\sqrt{1.6·10⁻⁷ m²/s·10⁻⁴ s}≈4 μm for an optional typical heating duration of, say, 100 μs . . . . In other words, during this 100 μs period, the heat generated in the local heating element by resistive heating has spread into the reaction solution surrounding the local heating element in approximately 4 μm.

The part of the reaction volume or reaction solution into which the heat can diffuse during the heating pulse is referred to below as the “heating zone” (AHZ). The extent of the heating zone perpendicular to the surface of the local heating element can be approximately estimated by the heat diffusion distance defined above.

Optionally, local heating of the reaction solution to the denaturation temperature by means of the local heating element is performed in such a manner that a heat diffusion distance into the reaction solution perpendicular to the surface of the local heating element is in a range of 0.05 μm to 200 μm.

Optionally, by suitable selection of the heating duration, only one or more partial volumes of the reaction solution are significantly heated, which optionally have expansions (measured perpendicularly from the surface of the microheater), i.e., heat diffusion width of optionally 0.05 μm to 200 μm, optionally 0.1 μm to 100 μm, optionally 0.1 μm to 50 μm, optionally 0.1 μm to 25 μm, optionally 0.1 μm to 15 μm, and optionally 0.1 μm to 10 μm. As used herein, the term “to be significantly heated” means to be significantly heated, but the temperature increase at the distance of a heat diffusion distance perpendicular from the surface of the local heating element is optionally less than 50 K, optionally less than 30 K, optionally less than 20 K, optionally less than 10 K, optionally less than 5 K.

Optionally, local heating of the reaction solution to the denaturation temperature by means of the local heating element is performed in such a manner that a temperature increase of the reaction solution at a distance from the local heating element which is twice the heat diffusion distance is not more than 5 K due to heating of the local heating element. On the one hand, sufficient spatial expansion of the heated area of the reaction solution perpendicular to the surface of the local heating element should ensure that the amplicons formed on the local heating element, which typically have a length of 0.02-3 μm (corresponding to approx. 60-10000 base pairs), can be heated as homogeneously as possible and thus denatured. On the other hand, the heat diffusion distance should optionally be sufficiently small to keep the volume ratio of the heating zone to the unheated passive volume of the reaction solution low.

Localization of the thermal field can be achieved by optionally selecting the heating duration or the duration of the denaturation step to be less than 20 ms, optionally less than 10 ms, optionally less than 5 ms, optionally less than 3 ms, optionally less than 2 ms, optionally less than 1 ms. Accordingly, optionally, a heating duration of the local heating element for locally heating the reaction solution to the denaturation temperature is not more than 20 ms per denaturation step.

Optionally, the spatial spread of the temperature field can be controlled by selecting suitable heating durations. This is true in a non-equilibrium state, i.e., as long as no steady-state thermal gradient has formed, which in steady state depends only on the geometry of the heating device and the boundary conditions. Accordingly, local heating of the reaction solution to the denaturation temperature by means of the local heating element is optionally performed in such a manner that no stationary thermal gradient is generated in the reaction solution.

Due to the spatial propagation of heat according to the above equation, the amount of heat introduced into the reaction solution by the local heating element is distributed over an increasingly larger volume of the reaction solution, in such a manner that perpendicular to the surface of the local heating element, which is hotter by a temperature ATLocal (or also referred to as ΔT) than the global average temperature, a central temperature gradient of ΔT/d (ΔT/\sqrt{D·t}, wherein t here stands for the duration of the heating step) results, which enables heat transport.

This allows, for example, temperature gradients that optionally reach greater than 1K/μm, optionally greater than 3K/μm, and most particularly optionally greater than 5K/μm to achieve high localization of the temperature increase. Alternatively or additionally, the thermal gradients are optionally less than 1000K/μm and particularly optionally less than 300K/μm. This can be advantageous to avoid thermophoretic effects in the reaction solution.

A more accurate estimation of the spatial heat propagation during and after the heating pulse for a given geometry of the local heating element can be achieved, for example, by finite element methods, such as with commercial solutions like COMSOL, which allow a numerical solution of the heat diffusion equation.

Optionally, the PCR is performed in such a manner that in at least one of the runs of the amplification cycle of the PCR, the heating device or local heating element(s) supplies less heat generated in the denaturation step to the reaction volume or reaction solution than CR * 5° C., and wherein CR is the heat capacity of the reaction volume during heating by the heating device, and no temporally stable temperature gradient is established during the entire denaturation step to at least 10%, optionally, however, over the entire contact area of the heating device or of the local heating element or elements with the reaction volume, i.e. a non-equilibrium state is present.

A temperature gradient is considered “stable over time” within the meaning of the present disclosure after a duration t1 following the start of heating by the local heating element if the magnitude of its maximum slope at a time 2t1 has changed by less than 30% compared to the magnitude of its maximum slope at time t1.

Here, only the comparison of the amounts of the maximum slope is relevant for determining the temporal stability, but not whether the heating device or the local heating element(s) generates heat at the time 2t1 or not. Optionally, the amount of the slope at time 2t1 has changed by less than 20%, further optionally by less than 15%, more optionally by less than 10%, especially optionally by less than 5% compared to the amount of its maximum slope at time t1. The gradient usually has its maximum slope at the surface of the local heating element(s).

While some geometries allow steep temperature gradients to be realized even in steady-state equilibrium, local heating allows heating with particularly little energy by taking advantage of non-equilibrium states, i.e., the time of the heating process before a temporally stable temperature gradient has even formed. While in steady state equilibrium particularly steep temperature gradients can occur when there is a strong outflow of heat and thus energy, when non-equilibrium states are used a strong temperature gradient can be achieved with very small amounts of energy even when the outflow of heat from the reaction volume is small. Thus, local heating allows steep temperature gradients and, accordingly, rapid heating of the heating zone without requiring significant energy input and associated heating of the entire reaction solution.

One advantage of the short heating and denaturation steps is that the amount of energy required in the denaturation step is so small that active cooling (to return to the elongation or annealing temperature) can be dispensed with, since the small amount of heat input can be dissipated in the reaction volume and its surroundings. I.e. according to one embodiment, cooling from the denaturation temperature to the elongation or annealing temperature is performed passively, by heat diffusion, without active cooling, i.e. optionally without a special cooling device.

Denaturation and optionally also other steps of the nucleic acid amplification or PCR can thus take place locally in the direct vicinity of the local heating elements, wherein optionally at least one of the required primers is functionalized to the magnetic microparticles and by means of this is added to the heating zone near the heating device or the local heating element or to one of the local heating elements, in order to also allow the amplicon to form there and thus enable denaturation with local heating. In other words, heating of the reaction volume can be limited to a fraction of the reaction volume by optionally localizing steps of the PCR, in particular hybridization, elongation and/or denaturation, and optionally also generating a signal for monitoring the progress of the PCR in the immediate vicinity of the local heating element, due to the attachment of the microparticles to the local heating element.

The combination of a method for extracting a target nucleic acid and/or for amplifying a target nucleic acid according to one embodiment can thus be combined in a particularly advantageous manner with a device for performing PCR based on local heating. A particular advantage can result from the fact that the target nucleic acid is already arranged on the local heating element by means of the attached magnetic microparticles and the target nucleic acid is therefore already in the heating zone, which can be heated to the denaturation temperature or beyond by means of local heating. Particularly optionally, the magnetic microparticles are functionalized with primers for PCR, whereby it can be achieved that the target nucleic acids arranged on the heating element are already hybridized with a primer.

PCR optionally uses at least two oligonucleotides called “primers”, a forward primer and a reverse primer. The forward primer is complementary to the 3′ end of the original and the reverse primer is complementary to the 3′ end of the complement. In the hybridization step (also referred to as the “annealing step”), the forward primer and/or the reverse primer hybridizes to a sequence complementary to it in the original or complement or amplicon, respectively. The hybridization step usually occurs at a temperature that induces or at least promotes hybridization of the forward and reverse primers to their complementary sequences in the original and complement or amplicon, respectively. It is optionally chosen to allow the most specific hybridization of the primers. The hybridization temperature is typically between 50° C. and 72° C. One or more of the primers may optionally be functionalized to the magnetic microparticles. Optionally, either the forward primers or the reverse primers may be functionalized to the magnetic microparticles. Alternatively or additionally, at least some magnetic microparticles may also be functionalized with forward and reverse primers.

In the elongation step, the hybridized primers are complementarily extended by a polymerase enzyme. Thus, starting from the forward primer, a complement can be synthesized and starting from the reverse primer, an original can be synthesized. The polymerase is exposed to a temperature that allows or at least promotes elongation for the purpose of elongation. When using a polymerase from the bacterium Thermus aquaticus (Taq), an elongation temperature of 72° C. is typically used. In some embodiments of PCR, the hybridization and elongation temperatures are identical, that is, both steps occur at the same temperature (that is, there are only two temperature steps during PCR, a combined hybridization and elongation temperature and a denaturation temperature).

The denaturation temperature corresponds to a temperature at which a nucleic acid double strand is denatured, i.e. at which the nucleic acid double strand is separated into its two single strands. For example, the denaturation step may separate the extraction nucleic acid from the nucleic acid hybridized to it. An optional type of denaturation is thermal denaturation (also referred to as “melting”). For this purpose, at least a part of the nucleic acid double strand or the whole double strand is exposed to a temperature which is equal to or higher than the denaturation temperature and which causes or at least promotes a separation of the nucleic acid double strands. On the one hand, the optional denaturation temperature is selected high enough that nucleic acid double strands can be separated. On the other hand, the optional denaturation temperature is chosen low enough to avoid significant damage to a DNA polymerase that may also be present in the reaction solution. For example, a typical value for the denaturation temperature may be 95° C. Heating the local heating element to a temperature equal to or greater than the denaturation temperature may optionally provide the advantage of allowing the target nucleic acid to at least partially detach from the extraction nucleic acid and freely transfer into the reaction solution. This can be advantageous, for example, for subsequent amplification of the extracted nucleic acid and/or if the target nucleic acid is to be removed or separated from the local heating element.

Optionally, the method of extracting the target nucleic acid onto the sample fluid further comprises removing the sample fluid from the reaction container and providing an extraction solution in the reaction container in such a manner that the magnetic microparticles attached to the extraction element with the target nucleic acid hybridized thereto are at least partially suspended in the extraction solution. The fact that the magnetic microparticles and the associated target nucleic acids are attached to an extraction element, optionally for example to a container wall of the reaction container, means that they remain in the reaction container, while the remainder of the sample fluid that is no longer required can be removed from the reaction container. By filling the extraction solution, the magnetic microparticles sedimented on the container wall can be re-suspended and are thus ready for further use in the extraction solution. In order to achieve reliable suspension of the magnetic microparticles in the extraction solution, it can be advantageous to support the loosening of the attached microparticles by applying mechanical force, for example by shaking and/or vibrating, e.g. by means of a vortex mixer, and/or by stirring, e.g. by means of a magnetic stir bar, and/or by applying ultrasound to the extraction solution and/or the reaction container. The mechanical force application can also optionally be used for a mixing of the sample fluid and/or reaction solution and or for the washing step. Furthermore, as an alternative or in addition to the mechanical application of force, a variable magnetic field can optionally be used for mixing the magnetic microparticles in the reaction solution and/or in the sample fluid.

Optionally, the reaction container is subjected to one or more washing steps between removing the sample liquid and providing the extraction solution in the reaction container . . . . It may be advantageous in that after removal of the sample fluid, any remaining sample fluid and/or other contaminants in the reaction container can be removed, thereby reducing or avoiding potential interference with the intended use of the extracted target nucleic acid.

Optionally, the extraction element is at least partially formed of ferromagnetic material. This optionally allows the magnetic field to be provided by means of an interaction of the ferromagnetic extraction element with the magnetic microparticles. Optionally, in such an embodiment, no further magnet is required to provide the magnetic field necessary for attaching the microparticles to the extraction element. Optionally, the extraction element has a foil and/or a wire, or is formed as a foil or a wire. Optionally, the extraction element is formed on a container wall of the reaction container and/or forms a part of a container wall of the reaction container.

Optionally, the method for amplifying the target nucleic acid further comprises exposing at least a part of the magnetic microparticles attached to the local heating element to a magnetic field in such a manner that the magnetic microparticles leave the locally heated area, i.e., the heating zone, of the reaction solution and are suspended in the reaction solution. In other words, a magnetic field or force is additionally used to re-suspend the magnetic microparticles deposited on the local heating element in the reaction solution in such a manner that they can optionally bind with further target nucleic acids or primers in the reaction solution and be elongated to generate a further amplicon. This is made possible in particular by the fact that during local heating in the area of the local heating element, while the magnetic microparticles are attached to it, the target nucleic acids hybridized to the primers of the magnetic microparticles are denatured and detach from the primers, whereupon the primers are again present in single-stranded form and are in principle available for hybridization with another target nucleic acid.

Optionally, the temperature of the reaction solution outside the locally heated area, i.e., the heating zone, is substantially the same as the hybridization temperature of the target nucleic acid. This offers the advantage of allowing hybridization of single-stranded primers with single-stranded target nucleic acids and elongation outside the locally heated or heating area. This can be particularly advantageous if, as described above, a magnetic field or force is used to repel the magnetic microparticles attached to the local heating element from the local heating element in such a manner that they are re-suspended in the reaction solution. The hybridization temperature can optionally represent a hybridization temperature range. For example, different individual hybridization temperatures can be used to amplify multiple different target nucleic acids in a reaction solution as part of multiplexing, wherein different target nucleic acids optionally have different hybridization temperatures, wherein the different hybridization temperatures are within the hybridization temperature range.

Optionally, when the area of the reaction solution in which the magnetic microparticles are attached is heated locally, the reaction solution outside the locally heated area remains substantially isothermal. “Substantially isothermal” in this context means that the reaction solution does not undergo temperature cycling outside the locally heated area and, in particular, does not reach the denaturation temperature. Optionally, the reaction solution remains outside the locally heated area in a temperature range between the optional annealing temperature and a temperature which is about 10° C. above the optional annealing temperature. This offers the advantage that no cooling of the reaction solution, in particular no active cooling device, has to be provided for cooling the reaction solution. In addition, this offers the advantage that the enzymes in the reaction solution do not have to be exposed to high temperatures or high temperature differences in large parts of the reaction solution.

Optionally, a device for amplifying the target nucleic acid is designed in such a manner that the temperature of the reaction solution outside the locally heated area is only passively reduced. In other words, the device is designed in such a manner that no active cooling of the reaction solution occurs, for example by means of a Peltier element and/or a cooling compressor. Rather, the reaction solution is cooled only by a natural release of heat to the environment, without the reaction solution being cooled by artificially inducing an increased temperature gradient between the reaction solution and its environment. This offers the advantage that no active cooling device, and in particular no power supply for an active cooling device, needs to be provided.

Accordingly, in a method for amplifying a target nucleic acid, the reaction solution is cooled only by a heat release to the environment of the reaction solution. There is no active cooling of the reaction solution and the direct environment of the reaction solution with which the reaction solution is in direct thermal contact.

Optionally, the method can be used to amplify several different target nucleic acids in one reaction solution. This allows multiplexing to be achieved. Optionally, different primers for different target nucleic acids can be attached to a microparticle. Alternatively or additionally, microparticles can be provided, each having only one type of primer for a target nucleic acid.

The denaturation temperature may optionally represent a denaturation temperature range. For example, different individual denaturation temperatures may be used to amplify multiple different target nucleic acids in a reaction solution as part of multiplexing, wherein different target nucleic acids optionally have different denaturation temperatures, wherein the different denaturation temperatures are within the denaturation temperature range.

Optionally, the reaction solution is provided in the reaction container in step f) in such a manner that the magnetic microparticles attached to the local heating element with the target nucleic acid hybridized thereto are at least partially suspended in the reaction solution. This offers the advantage that the microparticles can be distributed back into the reaction solution where they can optionally hybridize with additional target nucleic acids.

Optionally, step c) of the above-described method for amplifying a target nucleic acid comprises annealing at least a part of the sample fluid to the hybridization temperature of the target nucleic acid. This allows efficient hybridization of the single-stranded target nucleic acids or (reverse) primers present in the sample fluid with the single-stranded extraction nucleic acids or primers functionalized on the magnetic microparticles.

Optionally, between steps e) and f) of the above-described amplification method, the reaction container is subjected to one or more washing steps. This offers the advantage of removing any impurities and/or residues of the sample fluid that are undesirable for performing the subsequent amplification reaction.

Optionally, the area heated by the local heating element in step g) of the above-described amplification method, i.e., the heating zone, is heated to the denaturation temperature in a plurality of successive heating steps, respectively, and cooled to substantially the hybridization temperature between heating steps. “Cooled substantially to the hybridization temperature” in this context means that the optimum hybridization temperature need not be reached exactly, but rather that a temperature range may be sufficient in which hybridization and/or elongation can occur with sufficiently high efficiency. In particular, cooling to the hybridization temperature can occur independently, by diffusion of the heat introduced during heating from the heated area into the remaining reaction solution, which in this manner serves as a heat reservoir or cold reservoir. It can be advantageous if the remaining reaction solution is kept substantially at the hybridization temperature, for example by means of an external heating block.

Optionally, the target nucleic acid is amplified by PCR in the area heated by the local heating element by means of the multiple heating steps and the cooling steps in between. This offers the advantage that cycling through the various temperature steps or thermal cycling can be performed in a particularly short time, since only a very short period of time is required for heating to the denaturation temperature and cooling to the hybridization temperature due to the use of local heating.

Optionally, the local heating element comprises or is configured as one or more electrically heatable metal foils. This offers the advantage that the local heating element can have a large surface-to-volume ratio and thus provide a large heating surface while maintaining a low heat capacity. This can both provide a large heatable area and minimize the time required for heating and cooling the heated area and local heating element, or its thermal inertia.

Optionally, the heated or heating area of the reaction solution by means of the local heating element has a temperature gradient extending away from the local heating element during heating, wherein the magnitude of the temperature gradient is optionally bisected along a length between 1 μm and 10 μm from the surface of the local heating element. The thickness of the heated area or the length over which the heated area extends from the local heating element into the reaction solution can optionally be selected in such a manner that the magnetic microparticles attached to the local heating element and the target nucleic acids bound thereto are located in this heated area, but the volume of the heated area is nevertheless as small as possible in relation to the volume of the remaining reaction solution, in order to provide as large a heat or cold reservoir as possible.

Optionally, the magnet is configured to generate a variable magnetic field in such a manner that, in a first state, the variable magnetic field acts on at least a part of the magnetic microparticles present in the reaction solution in such a manner that they attach to the local heating element and, in a second state, acts on the magnetic microparticles attached to the local heating element in such a manner that they leave the local heating element and are suspended in the reaction solution. This allows the magnetic microparticles to be optionally attached to and repelled from the local heating element multiple times, allowing them to hybridize with additional target nucleic acids in the reaction solution.

Optionally, the local heating element forms at least a part of a container wall of the reaction container. This offers the advantage that direct contact between the local heating element and the sample fluid or reaction solution can be established in a simple manner. Further, this offers the possibility to manufacture the device or reaction container with low manufacturing effort and thus keep the manufacturing costs low.

Optionally, the magnet or plurality of magnets has a permanent magnet and/or an electromagnet that can be changed in position and/or orientation relative to the reaction container. For example, when using a permanent magnet, the magnetic field can be changed by changing an orientation of the permanent magnet to the reaction container and/or a distance of the permanent magnet from the reaction container. The direction of the magnetic field can also be changed, for example, by reversing the permanent magnet relative to the reaction container in such a manner that, for example, the side of the permanent magnet facing the reaction container changes from the magnetic north pole to the south pole of the permanent magnet or vice versa. When using an electromagnet, for example, the variable magnetic field can be changed by changing the current flow, in such a manner as the current intensity and/or the direction of the current flow. For example, the electromagnet may comprise one or more solenoid coils and optionally a ferromagnetic core. Optionally, the magnet is formed on a side of the local heating element facing away from the reaction container. This offers the advantage that the permanent magnet in this arrangement makes it particularly effective and easy to attract the magnetic microparticles to the local heating element. Alternatively or additionally, the one or more magnets may be changed in position relative to the reaction container to provide a variable magnetic field in the reaction solution. Alternatively or additionally, several magnets with different polarity can be brought to the reaction container to achieve a variable magnetic field in the reaction solution.

Suitable parameters for performing an amplification reaction and, in particular, a PCR, particularly with regard to suitable ingredients of the reaction solution, can be taken, for example, from the DE102016120124A1 publication. Also, exemplary data can be taken from DE102016120124A1 with respect to temperatures and time durations for local heating, in such a manner that reference is made in this respect to the already published publication and the disclosure of said publication is to be regarded as comprised by the disclosure of the present disclosure.

The above-mentioned features and embodiments and examples explained below are to be regarded not only as disclosed in the combinations explicitly mentioned in each case, but are also comprised by the disclosure content in other technically useful combinations and embodiments.

Further details and advantages will now be explained in more detail with reference to the following examples and optional embodiments with reference to the figures.

In the drawings:

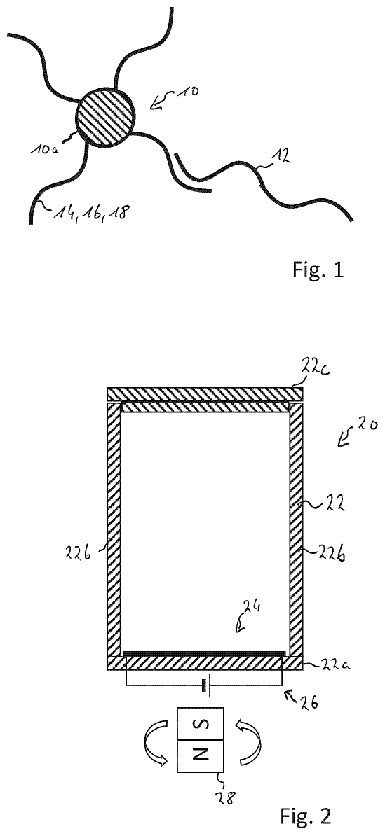

FIG. 1 is a functionalized magnetic microparticle according to an optional embodiment;

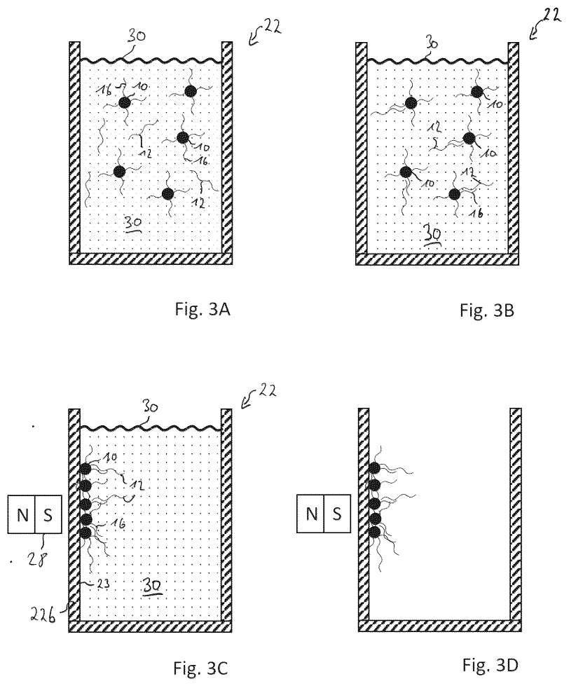

FIG. 2 is a schematic representation of a reaction container according to an optional embodiment;

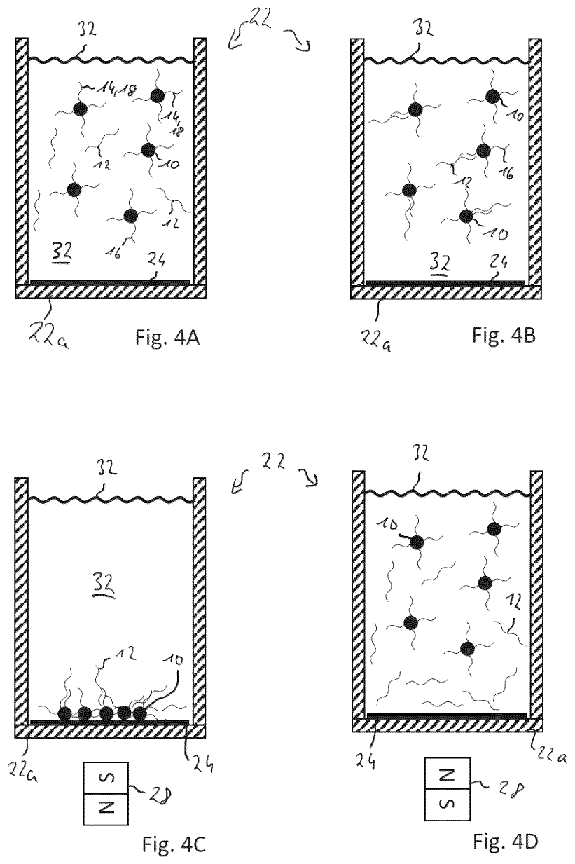

FIGS. 3A to 3D are explanations of a method for extraction of a target nucleic acid according to an optional embodiment;

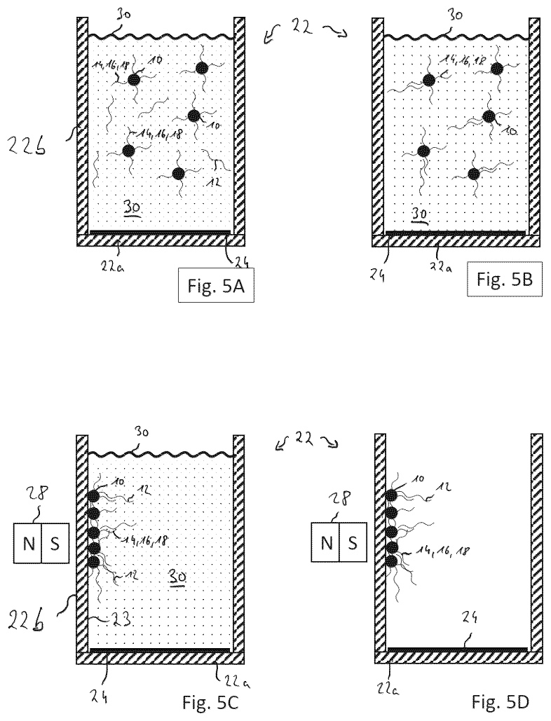

FIGS. 4A to 4D are explanations of a method for amplification of a target nucleic acid according to an optional embodiment;

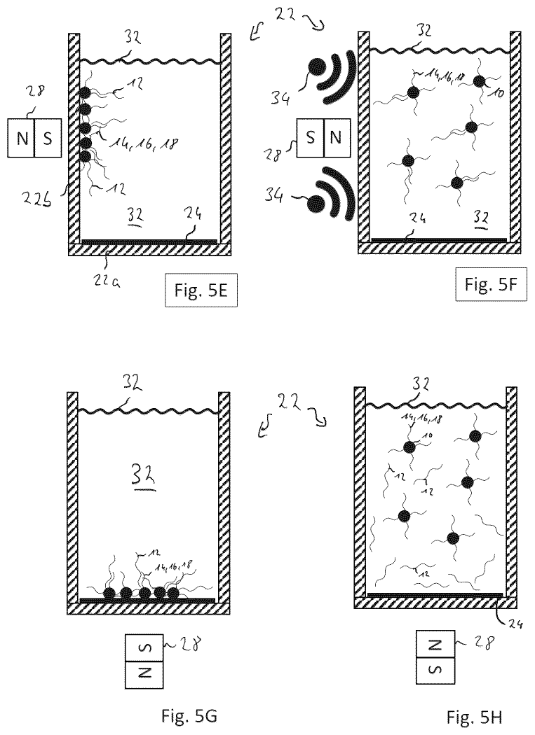

FIGS. 5A to 5H are explanations of a method for extraction and amplification of a target nucleic acid according to an optional embodiment;

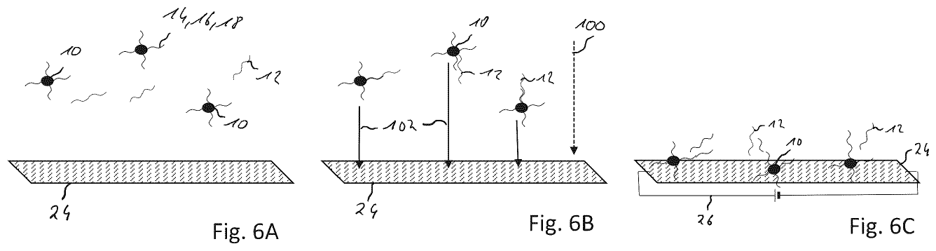

FIGS. 6A to 6C are explanations of a working principle of amplification of a target nucleic acid according to an optional embodiment.

In the following figures, the same or similar elements in the various embodiments are designated with the same reference signs for the sake of simplicity.

FIG. 1 shows a schematic representation of a magnetic microparticle 10 that can be used in a method for extraction and/or amplification of a target nucleic acid 12 according to an optional embodiment. In this regard, the magnetic microparticle 10 is formed at least in part from a ferromagnetic material, thereby providing the magnetic properties of the microparticle. The microparticle 10 is spherical in shape according to the optional embodiment shown, although other shapes are possible according to other embodiments. The size of the microparticle or its diameter is in an area of 1 μm.

In addition, according to the optional embodiment shown, the microparticle 10 has a coating 10a that enables or facilitates functionalization of the microparticle 10 with nucleic acids. In this regard, the coating may consist of or comprise streptavidin or biotin, thereby enabling or facilitating functionalization of the microparticle with nucleic acids using a streptavidin-biotin compound. Optionally, streptavidin is attached to the microparticle and biotin to the primers intended for functionalization, although an opposite arrangement is also possible.

In the schematic diagram shown, four oligonucleotides 14 can be identified which are functionalized to the magnetic microparticle 10. For clarity, only four oligonucleotides are shown, although the actual occupation density of the surface of the microparticle with oligonucleotides may be significantly greater. Also, the actual size ratios of oligonucleotides 14 and nucleic acids in general to the microparticle 10 may differ significantly from that shown. For use of the microparticle 10 in a method for extraction of a target nucleic acid 12, the oligonucleotides may be extraction nucleic acids 16. For use in a method of amplifying a target nucleic acid 12, the oligonucleotides 14 may be primers 18. According to optional embodiments of methods that serve to extract and amplify a target nucleic acid 12, the oligonucleotides 14 may be formed as primers 18 and simultaneously have the function of an extraction nucleic acid 16. The oligonucleotides 14 may optionally have two or more portions. For example, a first portion may be formed as a functionalization portion by means of which the oligonucleotide is functionalized to the microparticle 10. A second portion may provide functionality of the oligonucleotide 10 as a primer 18 and/or extraction nucleic acid 16.

In the embodiment shown, a target nucleic acid 12 is hybridized or bound to one of the oligonucleotides 14. In this manner, the target nucleic acid 12 is firmly attached to the magnetic microparticle 10 via the oligonucleotide 14 and, in particular, remains attached to the microparticle 10 even when the microparticle 10 is removed from a sample fluid. In contrast, the target nucleic acid 12 can thereby be removed again from the oligonucleotide 14 and thus from the microparticle 10 by using denaturation to dissolve the hybridization of the target nucleic acid 12 with the oligonucleotide.

FIG. 2 shows a schematic representation of a device 20 according to an optional embodiment for amplifying a target nucleic acid 12. In this regard, the device 20 comprises a reaction container 22, a local heating element 24, and a magnet 26. The reaction container 22 again has a bottom 22a, a peripheral wall 22b or a plurality of walls 22b, and a lid 22c. According to the embodiment shown, the lid 22c is removable from the remainder of the reaction container 22, for example, to add or remove a sample fluid or a reaction solution from the reaction container 22. The reaction container 22 may be temperature-controllable independently of the local heating element 24, in such a manner as to bring or maintain a content therein at a hybridization temperature. The lid 22c can be temperature controlled separately from the rest of the reaction container 22, for example to avoid condensation of fluid on the interior side of the lid 22c. Optionally, the reaction container is at least partially transparent to allow visual inspection of the contents from the outside without opening the reaction container 22 and/or to allow optical measurement of the contents through the reaction container 22. Such an optical measurement can be advantageous, for example, for performing an amplification reaction, particularly when combinations of dyes and quenchers are used to detect the amplicons and successful amplification is performed based on an optical signal from these dyes.

The local heating element 24 is optionally formed as a metal foil, which can be resistively warmed or heated by the application of electric current. The metal foil is optionally made as thin as possible in order to have a low volume and thus a low heat capacity despite a large heating surface. The local heating element according to the embodiment shown has a voltage source 26 for the power supply for heating the metal foil, by means of which electrical energy can be supplied to the metal foil . . . . Optionally, the local heating element or metal foil has one or more holes (not shown) that allow optical measurement of the contents in transmission through the reaction container 22 from the lid 22c through the interior or contents of the reaction container 22, through the hole or holes in the local heating element 24, and through the bottom 22a of the reaction container.

Furthermore, the device 20 has a magnet 28 which, according to the embodiment shown, is arranged below the reaction container 22 and in particular below the local heating element 24. By means of the magnet 28, a magnetic field can be provided in the interior of the reaction container 22 to attract magnetic microparticles 10 located in the reaction container 22 in such a manner that they attach to the local heating element 24, or repel them in such a manner that they detach from and move away from the local heating element 24. The magnet 28 may optionally be formed as or comprise a permanent magnet and may be changed in orientation relative to the reaction container 22 to reverse the polarity or change the magnetic field. Alternatively or additionally, the magnet 28 may comprise an electromagnet which provides a magnetic field of the desired strength and/or polarity by a change in current flow and the magnetic induction caused thereby. In this regard, it is advantageous if the reaction container 22 does not have ferromagnetic properties, at least in part, and optionally does not have diamagnetic properties, in order to allow penetration of a magnetic field generated outside the reaction container 22 without affecting it to a significant extent.

With reference to FIGS. 3A to 3D, a method according to an optional embodiment for extraction of a target nucleic acid 12 from a sample fluid 30 is explained below. For the sake of simplicity, the reaction container 22 is shown without a lid or other optional accessories in the schematic illustrations.

The reaction container 22 contains the sample fluid 30 in which magnetic microparticles 10 functionalized with extraction nucleic acid 16 and the single-stranded target nucleic acids 12 move freely. In addition, the sample fluid 30 may contain other elements and materials (not shown), but these must be separated from the target nucleic acid 12 during extraction. For example, the single-stranded target nucleic acids 12 present in the sample fluid 30 may have been introduced into the sample fluid by conventional methods, such as by extracting bacteria and/or viruses from a throat swab. Similarly, the sample fluid 30 may have been subjected to a lysis process and/or denaturation process prior to extraction or the target nucleic acids 12 may have been subjected to a lysis process and/or denaturation process in other environments prior to extraction to have the target nucleic acids 12 to be extracted present free and single stranded.

In a first step, shown in FIG. 3A, the magnetic microparticles 10 are thus provided in the sample fluid 30, in which the target nucleic acids 12 are also free and single-stranded.

FIG. 3B shows a second step in which at least a part of the target nucleic acids 12 hybridize to the extraction nucleic acids 16 of the microparticles 10. For this purpose, it can be advantageous if the sample fluid is tempered to the hybridization temperature, for example to about 60° C., in order to favor the hybridization process.

In the third step, which is shown in FIG. 3C, a magnetic field is provided inside the reaction container 22 or sample fluid 30. According to the embodiment shown, this is achieved by arranging a magnet 28 outside the reaction container 22, wherein the magnet 28 is arranged outside the left wall 22b of the reaction container in such a manner that the magnetic microparticles 10 present in the sample fluid 30 are at least partially attracted by it and attach to the wall 22b of the reaction container 22. The wall 22b of the reaction container 22 serves as the extraction element 23. According to other embodiments, a separate extraction element may also be provided, which may be placed, for example, in the reaction solution 32 and in the reaction container 22.

In the fourth step, shown in FIG. 3D, the sample fluid 30 was removed from the reaction container 22, but the magnetic microparticles 10 attached to the wall of the reaction container and the target nucleic acids 12 bound thereto were not also removed from the reaction container 22. The sample fluid can be removed from the reaction container 22, for example, by pouring it out and/or sucking it out of the reaction container 22, wherein a suction effect that is as low as possible is advantageous in order to avoid, as far as possible, loosening and removal of the microparticles 10 attached to the reaction container 22. Depending on the strength of the adherence of the sedimented magnetic microparticles 10 to the reaction container 22 and on the suction effect occurring during removal of the sample fluid 30, it is not mandatory to maintain the magnetic field, since the adhered microparticles optionally remain in the adhered state even without a magnetic field.

Also, it is not mandatory that the microparticles attach to a side wall 22b of the reaction container 2. Also, if the magnetic field is appropriate, attachment can occur to another boundary of the reaction container 22, such as the bottom 22a or the interior side of the lid if it is in contact with the sample fluid 30.

Thus, this optional method extracted the target nucleic acids 12 from the sample fluid 30 using the magnetic microparticles. The target nucleic acids 12 are thereby available for further use. For example, these may be re-detached from the container wall 22b by introducing a different fluid into the reaction container 22 and re-suspended in the fluid.

With reference to FIGS. 4A to 4D, a method according to an optional embodiment for amplification of target nucleic acids 12 is explained below. Amplification of the target nucleic acids 12 occurs in a reaction container 22, which has a local heating element 24 at the bottom 22a. In this case, the local heating element 24 is formed as a metal foil which extends over the bottom 22a of the reaction container 22 on the interior side of the reaction container 22.

The reaction container 22 is filled with a reaction solution 32 (FIG. 4A). The reaction solution 32 is adapted to perform an amplification reaction for the target nucleic acid 12, in particular by means of a PCR. To this end, the reaction solution 32 may include, in particular, the ingredients necessary for PCR, such as the nucleotides and/or enzymes and/or ingredients of a buffer solution, as well as other primers that are different from the primers 18 functionalized to the magnetic microparticles 10. For example, if the primers 18 functionalized to the magnetic microparticles 10 are forward primers, there may be freely movable reverse primers in the reaction solution 32. Where the primers 18 on the magnetic microparticles 18 are formed as reverse primers, the additional primers present in the reaction solution 32 may be formed as forward primers. Both the target nucleic acid 12 and the magnetic microparticles 10 are suspended and freely movable in the reaction solution 32. The oligonucleotides 14 are thereby formed as primers 18, wherein the primers 18 are at least partially complementary to a sequence portion of the target nucleic acid 12 and serve as primers in the amplification reaction.

The reaction solution 32 is provided at conditions that allow hybridization of the target nucleic acids 12 to the primers 18 functionalized to the magnetic microparticles 10 (FIG. 4B) and elongation of the primers by a polymerase provided in the reaction solution 32. In particular, these conditions may comprise tempering the reaction solution 32 to a hybridization temperature. In addition, the conditions may comprise further chemical and/or biological conditions that, in particular, allow the target nucleic acids 12 to be present in single-stranded form. Those target nucleic acids 12 which are hybridized with a primer 18 are then at least partially elongated by a polymerase to form a double strand, thereby generating amplicons of target nucleic acid 12 which are then initially bound to the magnetic microparticle 10 as a double strand with the target nucleic acid 12.

After hybridization and amplification, a magnetic field is created in the reaction container 22 or in the reaction solution 32 by means of a magnet 28, by means of which the magnetic microparticles 10 and the target nucleic acids 12 bound to them are moved to the local heating element 24, in such a manner that they are at least partially deposited there (FIG. 4C). In this manner, a concentration of the magnetic microparticles 10 and, consequently, a concentration of the bound target nucleic acids 12 occurs at the local heating element 24 and thus in the area of the reaction solution 32 that is locally heated to the denaturation temperature by the local heating element 24. Since only a very limited area of the reaction solution with a very small volume needs to be heated to the denaturation temperature, the heating and subsequent cooling back to the hybridization temperature can be done in a very short period of time and with a very low heat input. Heating the area in which the target nucleic acids 12 are located to the denaturation temperature causes them to denature and consequently detach from the primers 18 functionalized to the magnetic microparticles 10. Thus, the now again single-stranded target nucleic acids 12 and the again single-stranded (elongated) primers 18 functionalized to the magnetic microparticles 10 are ready for further amplification reactions.

To promote re-hybridization and amplification of the now elongated primers 18 on the magnetic microparticles 10 in the reaction solution 32, in a further optional step, the magnetic microparticles 10 are repelled from the local heating element 24 by providing an appropriate magnetic field in the reaction solution 32 in such a manner that they are again suspended in the reaction solution 32 and are again freely mobile in the reaction solution 32. Alternatively or additionally, a mechanical force may optionally be provided to at least partially re-suspend the microparticles 10 attached to the local heating element 24 in the solution. Since the reaction solution 32 is optionally and particularly maintained at the hybridization temperature outside the heated area at the local heating element 24, this promotes re-hybridization of the primers 18 and subsequent elongation. In particular, this may involve hybridization of primers 18 with primers different therefrom that are freely present in reaction solution 32 and subsequent elongation by a polymerase to form another double strand to again generate an amplicon of target nucleic acid 12. In order to separate this double strand again, an appropriate magnetic field can be provided again, by means of which the magnetic microparticles 10 are moved to the local heating element 24, where they are heated to the denaturation temperature. These steps can be repeated as many times as desired in such a manner that a polymerase chain reaction is thereby performed and exponential amplification of the target nucleic acids 12 originally present in the reaction solution 32 is obtained. The different magnetic fields, which are required on the one hand for attracting the magnetic microparticles 10 to the local heating element 24 and on the other hand for repelling the magnetic microparticles from the local heating element 24, can be provided, for example, by a permanent magnet which can be changed in its orientation relative to the reaction container 22. Alternatively or additionally, the magnet 28 may have one or more electromagnets, which may also generate a variable or reversible magnetic field.

According to another optional embodiment, the magnetic microparticles are attracted to the local heating element 24 only once by means of a magnetic field provided for this purpose, in such a manner that the magnetic microparticles 10 accumulate there. Repulsion of the microparticles 10 from the local heating element 24 does not occur according to this embodiment. Rather, the amplification of the target nucleic acid is accomplished by heating the heating zone locally to the denaturation temperature several times, with the temperature in the heating zone dropping back to the annealing or hybridization temperature between denaturation steps. Removal of the attached microparticles from the local heating element is therefore not required for temperature cycling at the local heating element.

With reference to FIGS. 5A to 5H, a method according to an optional embodiment for extraction and amplification of a target nucleic acid 12 is explained below.

The method thereby comprises both an extraction of the target nucleic acids 12 from a sample fluid 30 and amplification of the extracted target nucleic acids 12 in a reaction solution. According to the optional embodiment shown, both the extraction of the target nucleic acids 12 from the sample fluid 30 and the amplification of the target nucleic acids 12 in the reaction solution are performed in the same reaction container 22.

For this purpose, the reaction container 22 is equipped with a local heating element 24 on the interior side of its bottom 22a, which is not required for the extraction but is later used for amplification of the extracted target nucleic acids 12. For the sake of clarity, the power source or voltage source by means of which the local heating element 24 can be heated is not shown.

The extraction of the target nucleic acids 12 from the sample fluid 30 is shown in FIGS. 5A to 5D and corresponds substantially to the method according to the optional embodiment which has already been explained with reference to FIGS. 3A to 3D. It should be noted that during the extraction of the target nucleic acids 12 or magnetic microparticles 10, they are attached or sedimented to a side wall 22b, but not to the local heating element 24, in such a manner that the side wall 22b serves as the extraction element 23. This offers the advantage that any co-extracted impurities are not deposited on the local heating element 24 during extraction and may interfere with or impair the subsequent amplification reaction. Rather, this allows the extraction of the magnetic microparticles 10 under target nucleic acids 12 to be initially separated from the amplification reaction. Also, according to this method, the oligonucleotides 14 functionalized to the magnetic microparticles 10 are formed as primers 18 for the subsequent amplification reaction and have both the function of an extraction nucleic acid 16 and the function of a primer 18.

In FIG. 5D, the extracted magnetic microparticles 10 and associated target nucleic acids 12 are present attached to a sidewall 22b of the reaction container 22, wherein the sample fluid 30 has been removed from the reaction container and the reaction container 22 has optionally been subjected to one or more washing steps to remove any unwanted residues from the reaction container 22.

The subsequent amplification method comprises amplification of the target nucleic acids 12 in a reaction solution 32, which is explained with reference to FIGS. 5E to 5H and is substantially the same as the amplification method explained above with reference to FIGS. 4A to 4D.

First, the reaction solution 32 is filled into the reaction container 22, wherein the reaction solution 32 is designed in such a manner that it enables the amplification reaction, for example a PCR, to be performed (FIG. 5E).

In a further step, shown in FIG. 5F, the magnetic microparticles 10 attached to the side wall 22 B of the reaction container 22 are then dissolved in such a manner that they are at least partially suspended in the reaction solution 32. This can be done by reversing the polarity of the magnet 28 in such a manner that the magnetic field generated by the magnet 28 no longer moves the magnetic microparticles 10 to the side wall 22, but repels them from it. Alternatively or additionally, a mechanical force can be applied to the magnetic microparticles 10, for example by stirring the reaction solution 32 and/or by acting on the reaction container 22 and/or the reaction solution 32 by means of ultrasonic impact, as exemplified by the ultrasonic waves 34 shown symbolically in FIG. 5F. This results in the magnetic microparticles 10 with their associated target nucleic acids 12 being suspended in the reaction solution and free in the reaction solution 32. By having in the reaction solution 32 all the ingredients necessary to perform the amplification reaction, such as in particular enzymes and the primers complementary to the primers 18, and by maintaining the reaction solution 32 at approximately the hybridization temperature, elongation of the primers 18 occurs.

In a further step, which is shown in FIG. 5G, the magnetic microparticles 10 in the reaction solution 32 are moved to the local heating element 24 by means of a magnetic field, in such a manner that they sediment there at least in part. This can be done, for example, by arranging a magnet 28 below the reaction container 22 and, in particular, below the local heating element 24 in such a manner that the magnetic microparticles 10 are attracted by the magnets and are deposited accordingly on the local heating element 24. This results in the magnetic microparticles 10 and the target nucleic acids 12 associated therewith being located in the area which is locally heated by the local heating element 24, i.e. in the heating zone, in such a manner that the denaturation temperature is reached or exceeded and, accordingly, the target nucleic acids 12 hybridized to the optionally completed primers 18 separate and are again in free solution.

Furthermore, in a further optional step, as shown in FIG. 5H, the magnetic microparticles 10 are now repelled from the local heating element 24 by means of the magnet 28, in such a manner that they are suspended again in the reaction solution 32, and can hybridize in the reaction solution 32 with reverse primers and in this way, by means of a multiple run through of the steps shown in FIGS. 5G and 5H, can achieve an exponential amplification of the target nucleic acid 12 or of the generated amplicons. Optionally, in each duplication step, the magnetic microparticles 10 can be moved to the local heating element 24 by means of the magnet 28, heated there to the denaturation temperature by local heating by means of the local heating element 24 and, after denaturation, removed from the local heating element 24 again by reversing the polarity of the magnet 28 in order to hybridize again with reverse primers and/or target nucleic acids 12 in the reaction solution 32 tempered to the hybridization temperature.

For example, detection of the generated amplicons can be performed by optical means. For this purpose, for example, primers can be used which are formed with a dye and a quencher and only provide a fluorescence signal when these have become part of an amplicon by a polymerase and the dye has thus been separated from the quencher. To achieve optical detection, it may be advantageous if the reaction vessel 22 is at least partially transparent to the fluorescence wavelength of the dye and an excitation wavelength intended therefor. For example, the walls 22b, the bottom 22a and/or the lid 22c of the reaction container 22 may be transparent. Also, a measurement can be made from the top through the lid 22c vertically down through the reaction solution 32 and through the local heating element 24 the bottom 22a. To this end, it may be advantageous for the local heating element 24 to have one or more recesses through which the light for detecting the amplicons can at least partially pass to be detected below the reaction container is 22.

Suitable parameters for performing an amplification reaction and, in particular, a PCR, particularly with regard to suitable ingredients of the reaction solution, can be taken, for example, from the DE102016120124A1 publication. Also, DE102016120124A1 provides examples of the temperatures and durations for local heating, in such a manner that reference is made to the previously published publication.

With reference to FIGS. 6A to 6C, the principle of an amplification according to an optional embodiment is again explained schematically. Here, the embodiments are substantially reduced to the local heating element 24, the magnetic microparticles 10, and the target nucleic acids 12.

In FIG. 6A, the magnetic microparticles 10 and the target nucleic acids 12 are suspended and freely mobile in the reaction solution (not shown), wherein conditions prevail such that hybridization of the target nucleic acids 12 to the oligonucleotides 14 with which the magnetic microparticles are functionalized is enabled and occurs.

FIG. 6B shows a step in which a magnetic field is provided, symbolized by the arrow 100. The magnetic field 100 exerts a motion 102 on the magnetic microparticles 10, causing them to move the direction of the arrow 102 toward the local heating element 24 and accumulate there.

FIG. 6C shows a further step in which the magnetic microparticles 10 are attached to the local heating element 24 and the area of the reaction solution in which the attached microparticles 10 are located is locally heated by means of the local heating element 24 in such a manner that the denaturation temperature is reached or exceeded in the area and the target nucleic acids 12 hybridized to the oligonucleotides 14 separate from the oligonucleotides 14.

Subsequently, the microparticles 10 can be repelled from the local heating element if necessary, for example by a magnetic field which now acts in the opposite direction, in such a manner that the microparticles 10 are again suspended in the reaction solution and disperse there to be available again for hybridization.

### LIST OF REFERENCE SIGNS

10 magnetic microparticles
10a coating of the microparticle
12 target nucleic acid
14 oligonucleotide
16 nucleic acid extraction
18 primer
20 device for amplifying a target nucleic acid
22 reaction container
22a bottom of the reaction container
22b reaction container wall
22c reaction container lid
23 extraction element
24 local heating element
26 voltage source
28 magnet
30 sample liquid
32 reaction solution
34 ultrasonic waves

1. A method for amplifying a target nucleic acid (12), the method comprising the steps of:
a) providing a sample fluid containing the target nucleic acid (12) in a reaction container (22) and at least one local heating element (24) in direct contact with the sample fluid (30);
b) providing magnetic microparticles (10) in the sample fluid (30), wherein the magnetic microparticles (10) are each functionalized with at least one primer (18) for amplifying the target nucleic acid (12);
c) hybridizing the target nucleic acid (12) with at least one of the primers (18) functionalized on the magnetic microparticles (10);
d) providing a magnetic field in the reaction container (22) in such a manner that at least a part of the magnetic microparticles (10) with the target nucleic acid (12) hybridized thereto attaches to the local heating element (24);
e) removing the sample fluid (30) from the reaction container (22);
f) providing a reaction solution (32) for performing an amplification reaction of the target nucleic acid (12) in the reaction container (22);
g) locally heating the reaction solution (32) to a denaturation temperature by means of the local heating element (24) in the area where the magnetic microparticles (10) are attached to the local heating element (24).

2. The method according to claim 1, wherein providing the reaction solution (32) in the reaction container (22) in step f) is performed in such a manner that the magnetic microparticles (10) attached to the local heating element (24) with the target nucleic acid (12) hybridized thereto are at least partially suspended in the reaction solution (32).

3. The method according to claim 1 or 2, wherein step c) comprises annealing at least a part of the sample fluid (30) to the hybridization temperature of the target nucleic acid (12).

4. The method according to any of claims 1 to 3, wherein between steps e) and f) the reaction container (22) is subjected to one or more washing steps.

5. The method according to any of claims 1 to 4, wherein the area heated in step g) by means of the local heating element (24) is heated to the denaturation temperature in a plurality of successive heating steps, respectively, and cooled substantially to the hybridization temperature between the heating steps.

6. The method according to claim 5, wherein amplification of the target nucleic acid (12) by means of PCR is performed by means of the plurality of heating steps and the intervening cooling steps in the area heated by means of the local heating element (24).

7. The method according to any of claims 1 to 6, wherein the local heating element (24) comprises one or more electrically heatable metal foils.

8. The method according to any of claims 1 to 7, wherein the area of the reaction solution (32) heated by means of the local heating element (24) has a temperature gradient extending away from the local heating element (24) during heating, and wherein the magnitude of the temperature gradient optionally bisects along a length between 1 μm and 10 μm from the surface of the local heating element.

9. The method for amplifying a target nucleic acid (12), the method comprising the steps of:
providing magnetic microparticles (10) in a reaction solution (32), each of which is functionalized with at least one primer (18) and is connectable or linked to at least one target nucleic acid (12) via the at least one primer (18);
providing a local heating element (24) in direct contact with the reaction solution (32);
exposing at least a part of the magnetic microparticles (10) associated with the target nucleic acid (12) in the reaction solution (32) to a magnetic field in such a manner that at least a part of the magnetic microparticles (10) attaches to the local heating element (24);
locally heating the reaction solution (32) to a denaturation temperature by means of the local heating element (24) in the area where the magnetic microparticles (10) are attached to the local heating element (24).

10. The method according to claim 9, further comprising the step of:
exposing at least a part of the magnetic microparticles (10) attached to the local heating element (24) to a magnetic field in such a manner that the magnetic microparticles (10) leave the locally heated area of the reaction solution (32) and are suspended in the reaction solution (32).

11. The method according to claim 10, wherein the temperature of the reaction solution (32) outside the locally heated area is substantially equal to the hybridization temperature of the target nucleic acid (12).

12. The method according to any of claims 9 to 11, wherein upon local heating of the area of the reaction solution in which the magnetic microparticles (10) are attached, the reaction solution outside the locally heated area remains substantially isothermal.

13. The method according to any of claims 9 to 12, wherein local heating of the reaction solution (32) to the denaturation temperature by means of the local heating element (24) is performed in such a manner that a heat diffusion distance into the reaction solution (32) perpendicular to the surface of the local heating element (24) is in an area of 0.05 μm to 200 μm.

14. The method according to claim 13, wherein the local heating of the reaction solution (32) to the denaturation temperature by means of the local heating element (24) is performed in such a manner that a temperature increase of the reaction solution (32) at a distance from the local heating element which is twice the heat diffusion distance is not more than 5 K due to the heating of the local heating element.

15. The method according to any of claims 1 to 14, wherein a heating duration of the local heating element (24) for locally heating the reaction solution (32) to the denaturing temperature is not more than 20 ms per denaturing step.

16. The method according to any of claims 1 to 15, wherein the local heating of the reaction solution (32) to the denaturation temperature by means of the local heating element (24) is performed in such a manner that no stationary thermal gradient is generated in the reaction solution.

17. The method according to any of claims 1 to 16, wherein the reaction solution is cooled only by a heat release to the environment of the reaction solution, and wherein no active cooling of the reaction solution and the immediate environment of the reaction solution with which the reaction solution is in direct thermal contact is performed.

18. The method of extracting a target nucleic acid (12) from a sample fluid (30), the method comprising the steps of:
providing a sample fluid (30) containing the target nucleic acid (12) in a reaction container (22);
providing magnetic microparticles (10) in the sample fluid (30), each functionalized with at least one extraction nucleic acid (16), wherein the extraction nucleic acids (16) are at least partially complementary to the target nucleic acid (12);
hybridizing at least a part of the target nucleic acid (12) with one of the extraction nucleic acids (16) and binding the target nucleic acid (12) via the nucleic acid extraction (16) to one of the magnetic microparticles (10); and
providing a magnetic field in the reaction container (22) in such a manner that at least a part of the magnetic microparticles (10) associated with the target nucleic acid (12) attaches to an extraction element (22) arranged in and/or on the reaction container.

19. The method according to claim 18, further comprising the steps of:
removing the sample fluid (30) from the reaction container (22) and/or removing the extraction element from the sample fluid (30); and
providing an extraction solution in the reaction container (22) and providing the extraction element in the extraction solution in such a manner that the magnetic microparticles (10) attached to the extraction element with the target nucleic acid (12) hybridized thereto are at least partially suspended in the extraction solution.

20. The method according to claim 19, wherein between removing the sample fluid (30) and providing the extraction solution in the reaction container (22), the reaction container (22) is subjected to one or more washing steps.

21. The method according to any of claims 18 to 20, wherein the extraction element is at least partially formed of ferromagnetic material.

22. The method according to any of claims 18 to 21, wherein the extraction element has a foil and/or a wire, or is formed as a foil or a wire; and/or
wherein the extraction element is formed on a container wall of the reaction container (22) and/or forms a part of a container wall (22) of the reaction container.

23. A device (20) for amplifying a target nucleic acid (12), the device (20) comprising:
a reaction container (22) adapted to receive a reaction solution (32) containing the target nucleic acid (12):
a local heating element (24) which is arranged in and/or on the reaction container (22) in such a manner that the local heating element (24) is at least partially in direct contact with the reaction solution (32) when the reaction container (22) is filled with reaction solution (32);
a magnet (28) for generating a magnetic field, wherein the magnetic field acts on at least a part of the magnetic microparticles (10) present in the reaction solution (32) in such a manner that they attach to the local heating element (24).

24. The device according to claim 23, wherein the magnet (28) is configured to generate a variable magnetic field in such a manner that, in a first state, the variable magnetic field acts on at least a part of the magnetic microparticles (10) located in the reaction solution (32) in such a manner that the magnetic microparticles (10) attach to the local heating element (24) and, in a second state, acts on the magnetic microparticles (10) attached to the local heating element (24) in such a manner that the magnetic microparticles (10) leave the local heating element (24) and become suspended in the reaction solution (32).

25. The device (20) according to claim 23 or 24, wherein the local heating element (24) is formed as or comprises a metal foil.

26. The device (20) according to any of claims 23 to 25, wherein the local heating element (24) forms at least a part of a container wall (22a, 22b, 22c) of the reaction container (22).

27. The device (20) according to any of claims 23 to 26, wherein the magnet (28) has a permanent magnet and/or an electromagnet variable in position and/or orientation relative to the reaction container (22).

28. The device (20) according to any of claims 23 to 27, wherein the magnet (28) is formed on a side of the local heating element (24) facing away from the reaction container (22).

29. The device (20) according to any of claims 23 to 28, wherein the device is configured in such a manner that the temperature of the reaction solution is only passively reduced and/or wherein the device does not have an active cooling device for cooling the reaction solution.

30. A use of magnetic microparticles (10) for extracting a target nucleic acid (12) from a sample fluid (30).

31. A magnetic microparticle (10) functionalized with at least one primer (18) for an amplification reaction of a target nucleic acid (12).
